(19) United States
(12) Patent Application Publication
(10) Pub. No.: US 2006/0211650 A1
(43) Pub. Date: Sep. 21, 2006

(54) Reducing carbohydrate derivatives of adamantane amines, and synthesis and methods of use thereof

(75) Inventors: Sebastian P. Assenza, Fort Salonga, NY (US); Nanzhu Shen, Medford, NY (US); Salvatore Iacono, Kings Park, NY (US); Yuriy B. Kalyan, Staten Island, NY (US); Peter Viski, Arlington, VA (US); Matthew Ronsheim, Port Jefferson, NY (US)

Correspondence Address: DARBY & DARBY P.C., P. O. BOX 5257, NEW YORK, NY, 10150-5257, US

(73) Assignee: Forest Laboratories, Inc., New York, NY (US)

(21) Appl. No.: 11/304,977

(22) Filed: Dec. 14, 2005

Related U.S. Application Data
(60) Provisional application No. 60/636,899, filed on Dec. 16, 2004.

(51) Int. Cl. A61K 31/7012; C07H 5/06

(52) U.S. Cl. 514/53; 536/18.7

(57) ABSTRACT

The present invention relates to reducing carbohydrate derivatives of adamantane amines of Formula A or pharmaceutically acceptable salts, solvates or derivatives thereof, wherein R₁, R₂, R₃, and R₄ are together or separately H, F, methyl or lower alkyl, alkenyl, or alkynyl groups, and Z is derived from a mono-, di-, oligo-, or poly-saccharide that originally had an aldehyde carbonyl group. The present invention also relates to processes for the preparation of such adamantane amine derivatives, and uses of such derivatives. The compounds of the present invention are useful in the treatment of infections caused by Gram positive or Gram negative bacteria.

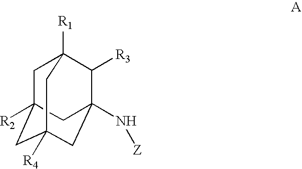

This application claims the benefit of U.S. Provisional Application No. 60/636,899 filed Dec. 16, 2004, which is hereby incorporated by reference.

### BACKGROUND OF THE INVENTION

This invention relates to adamantane amine derivatives formed by a reaction between reducing carbohydrates and adamantane amines.

Adamantane amines and their derivatives have long attracted attention due to their antiviral and neuroprotective properties. While there are numerous methods for the synthesis of adamantane amines with a free, primary amino group (U.S. Pat. No. 5,599,998, CN Patent No. 1,400,205, U.S. Pat. No. 3,388,164, and U.S. Pat. No. 3,391,142), subsequent alkyl derivatives thereof (U.S. Pat. No. 3,391,142), adamantine amide derivatives thereof (International Publication No. WO 03/068726) and metal complex derivatives thereof (International Publication No. WO 99/61450), no reports are available on reducing carbohydrate derivatives of adamantane amines. There are several publications on reducing carbohydrate derivatives of other primary amines besides adamantane amines. See Benson et al., Nucl. Acids Res. 28(1):15-18 (2000); R. Kuhn, L. Birkofer, Ber. 71B:621-35 (1938); Adachi, Susumu, Chemistry & Industry (1957); Shimamura et al., J. Agric. Food Chem. 48(12): 6227-29 (2000).

The primary product of a reaction between a primary amine and a reducing carbohydrate is usually an N-substituted carbohydrate amine. For example, lactosyl-amine (Formula B), which is produced from a reaction between lactose and 4-amino-toluene, or maltosyl-amine (Formula C), which is produced from a reaction between maltose and 4-amino-benzene-thiol.

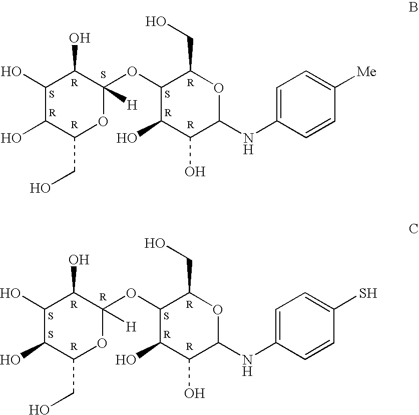

This type of reaction is called the Maillard reaction. Kramholler et. al, J. Agric. Food. Chem. 41(3):347-51 (1993); Shimamura et al., J. Agric. Food Chem. 48(12): 6227-29 (2000). Maillard reaction products can spontaneously, or upon treatment by heat or reagents, convert into the corresponding iso-amine (e.g., iso-lactosyl-amine (Formula D) or iso-maltosyl-amine (Formula E) via Amadori-rearrangement).

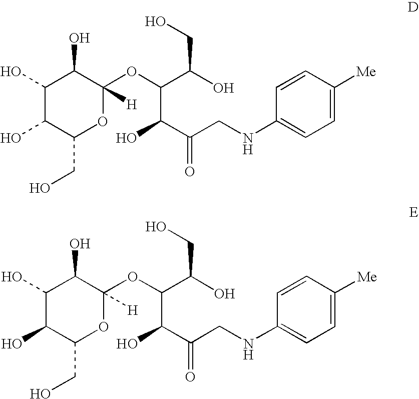

However, no similar compounds have been reported from adamantine amines and reducing carbohydrates.

### DETAILED DESCRIPTION OF THE INVENTION

The present invention describes derivatives of Formula A, 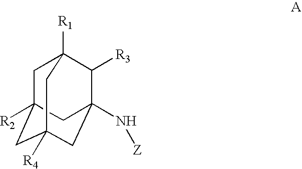
which are formed from adamantane amines and reducing carbohydrates, wherein R₁, R₂, R₃, and R₄ are together or separately H, F, methyl or lower alkyl, alkenyl, or alkynyl groups, and Z is represented by Formula F, 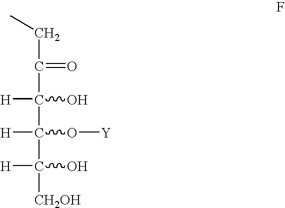
Formula F is a carbohydrate residue connected to Formula A via a methylene group next to the carbonyl group. Y can be hydrogen or a mono-, oligo-, or poly-saccharide. Z is derived from a mono-, di-, oligo-, or poly-saccharide that originally had an aldehyde carbonyl group, which is generally known as a “reducing carbohydrate.” Examples of such reducing carbohydrates are glucose, lactose, maltose, and the like.

The primary product represented by Formula G 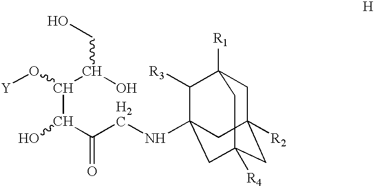
is the glicosyl-amine derivative of the adamantine amine, which spontaneously or artificially undergoes Amadori rearrangement in most cases, and thus forms the “iso-glicosyl” product of Formula H, wherein R₁, R₂, R₃, R₄, and Y are as defined above.

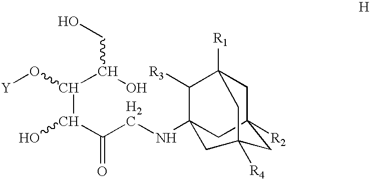

One specific embodiment of the present invention relates to such a derivative formed from memantine (3,5-dimethyl-adamantylamine) and lactose (Formula J).

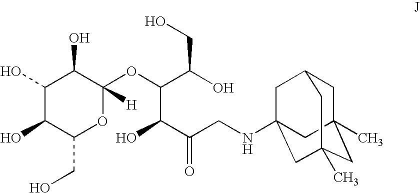

In a separate embodiment, the present invention provides methods for efficiently preparing such derivatives formed from adamantane amines and reducing carbohydrate derivatives. We have found that acetonitrile, dimethylformamide, dimethylacetamide, dimethylsulfoxide, and N-methylpyrrolidinone type solvents are especially advantageous as the reaction medium for the preparation of the present compounds, providing higher yields and more pure products than other solvents.

In another embodiment, the present invention provides methods of treating a patient suffering from an infection caused by Gram positive or Gram negative bacteria by administering an effective amount of a reducing carbohydrate derivative of an adamantane amine to a patient in need thereof. In U.S. Pat. No. 6,818,633, typical dosage amounts and administrative routes are provided for compounds having antiviral activity. Here, suitable dosage amounts will be in the range of 0.1 to 400 mg/kg of bodyweight of the recipient. Suitable administrative routes include, but are not limited to: oral, rectal, nasal, inhalation, topical, vaginal and parenteral.

### EXAMPLES

The instant invention provides a novel method for the preparation and purification of derivatives formed from adamantane amines and reducing carbohydrates, and is further described by means of the following examples. The use of these and other examples anywhere in the specification is illustrative only, and in no way limits the scope and meaning of the invention or of any exemplified form. Likewise, the invention is not limited to any particular preferred embodiments described herein. Indeed, modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

### Example 1

Memantine (3,5-dimethyl-adamantylatnine) free base (8.0 g; 0.045 mol) and lactose monohydrate (8.0 g.; 0.022 mol) were suspended in 65 ml of acetonitrile/water (1:1). The mixture was brought to reflux, initially forming a clear solution. Upon further heating to 3 hours, a dark brown suspension formed and heating continued for an additional hour. After cooling to room temperature, the mixture was concentrated to half volume and the mixture was extracted twice with 30 ml chloroform. The yellow colored aqueous solution was concentrated to a yellow gummy solid having a weight of 7.5 g. The LC/MS analysis of this material indicated 35-40% adduct product present.

The final purification of the lactose-memantine adduct product was separation of the crude material through preparative HPLC using ESLD (Evaporative Light Scattering Detection) on a Luna C18 column (Solvent A: water (0.1% TFA); Solvent B: acetonitrile (0.1% TFA)). The typical purity of isolated product was >99%.

### Example 2

A 250 mL two-neck round bottomed flask fitted with a condenser was charged with memantine (3,5-dimethyl-adamantylamine) free base (10.0 g, 27.9 mmol), lactose monohydrate (10.0 g, 55.8 mmol), DMF (80 mL), and water (1 mL). The mixture was heated at 73-78° C. for 18 hours. After this time, the mixture was allowed to cool to room temperature and concentrated by rotary evaporation. The residue was then dissolved in methanol (50 mL), and diethyl ether (500 mL) was added with stirring. The mixture was stirred for an additional 10 minutes. After this time, the precipitate was filtered and redissolved in methanol (50 mL). Diethyl ether (500 mL) was again added to the mixture and the resulting precipitate was filtered to afford the crude memantine-lactose adduct (7.0 g, 50%) as a red solid. The solid was dissolved in methanol (300 mL), and decolorizing carbon (10.5 g) was added. The mixture was allowed to stir at room temperature for 30 minutes, then filtered through celite. The filtrate was concentrated and the solid was placed in a vacuum oven for 5 hours to afford the memantine-lactose adduct (6.2 g) as a pale yellow solid of 91% HPLC purity. This product can be further purified by solid phase extraction.

### Example 3

### Antibacterial Properties of Memantine-Lactose Adduct

The antibiotic activity of a 1% memantine-lactose adduct solution in saline was assayed using a disk assay against three USP bacteria cultures: Staphylococcus aureus, Pseudomonas aeruginosa and Escherichia coli. A negative control, 0.9% saline disk, and a positive control, 1% gentamicin solution in saline, were included. Table 1 lists the results of the zone of inhibition test. Antibacterial activity was evaluated by measuring (in mm) the size of any clear zone of no growth (i.e., Zone of Inhibition) around each sample. A “No Zone” is reported when no antibacterial activity is observed. As indicated by Table 1, memantine-lactose adduct was surprisingly found to have antibacterial activity with respect to Staphylococcus aureus. These experiments may be predictive of biological effects in humans or other mammals and/or may serve as models for use of the present invention in humans or other mammals for the treatment of infections caused by Gram positive or Gram negative bacteria. See, e.g., Kustimur et al., Chinese Medical Journal, 116(4):633-636 (2003).

TABLE 1
Memantine-lactose Adduct Zone of Inhibition (mm) Test Results

| | | |
|---|---|---|
| Memantine-lactose | | |
| adduct, 1.0% | Negative control, | Positive control, 1% |
| solution | 0.9% saline solution | Gentamicin solution |

Staphylococcus aureus, ATCC #6538

| | | | |
|---|---|---|---|
| Plate 1 | No Zone | No Zone | 19.1 |
| Plate 2 | 9.1 | No Zone | 15.8 |
| Plate 3 | 8.3 | No Zone | 19.3 |

Pseudomonas aeruginosa, ATCC #9027

| | | | |
|---|---|---|---|
| Plate 1 | No Zone | No Zone | 11.2 |
| Plate 2 | No Zone | No Zone | 10.9 |
| Plate 3 | No Zone | No Zone | 9.9 |

Escherichia coli, ATCC #8739

| | | | |
|---|---|---|---|
| Plate 1 | No Zone | No Zone | 15.5 |
| Plate 2 | No Zone | No Zone | 15.5 |
| Plate 3 | No Zone | No Zone | 16.8 |

Definitions

As used herein, the term “reducing carbohydrates” includes all carbohydrates having an aldehyde end group, or possessing an acetal that in solution is in equilibrium with the free aldehyde form and their optical isomers, diastereomers, enantiomers, hydrates, pharmaceutically acceptable salts, and mixtures thereof.

As used herein, the term “lower” (e.g., “lower alkyl,” “lower alkenyl,” or “lower alkynyl”) refers to the corresponding radical group having 1-6 carbon atoms.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety and to the same extent as if each reference was individually incorporated by reference.

What is claimed is:

1. A compound of Formula (I) 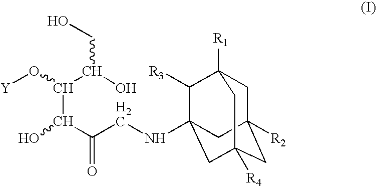
wherein R₁, R₂, R₃, and R₄ are together or separately H, F, methyl or lower alkyl, alkenyl, or alkynyl groups, and Y is selected from hydrogen and a mono-, di-, oligo-, or poly-saccharide or a pharmaceutically acceptable salt, solvate or derivative thereof.

2. A compound according to claim 1, wherein the compound has undergone an Amadori rearrangement from a compound represented by Formula (II) 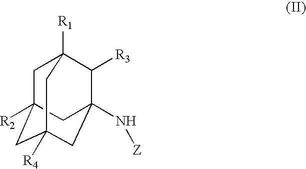
wherein R₁, R₂, R₃, R₄ are together or separately H, F, methyl or lower alkyl, alkenyl, or alkynyl groups, and Z is represented by Formula (III) 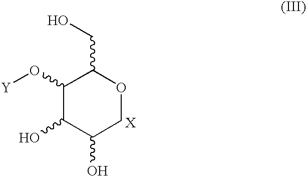
wherein X is the point at which the carbohydrate residue is connected to the nitrogen atom of Formula (II), and Y is hydrogen or a mono-, di-, oligo-, or poly-saccharide.

3. A compound of Formula (IV)

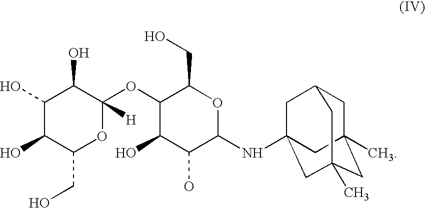

4. A compound of Formula (V)

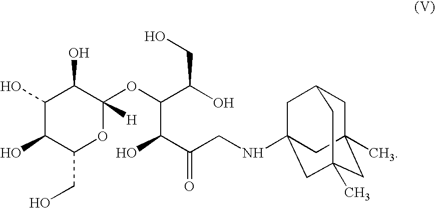

5. A method for preparing a compound of claim 1, wherein the reaction medium comprises a solvent selected from acetonitrile, dimethylformamide, dimethylacetamide, dimethylsulfoxide and N-methylpyrrolidinone.

6. A method for preparing a compound of claim 2, wherein the reaction medium comprises a solvent selected from acetonitrile, dimethylformamide, dimethylacetamide, dimethylsulfoxide and N-methylpyrrolidinone.

7. A method for preparing a compound of claim 3, wherein the reaction medium comprises a solvent selected from acetonitrile, dimethylformamide, dimethylacetamide, dimethylsulfoxide and N-methylpyrrolidinone.

8. A method for preparing a compound of claim 4, wherein the reaction medium comprises a solvent selected from acetonitrile, dimethylformamide, dimethylacetamide, dimethylsulfoxide and N-methylpyrrolidinone.

9. A method of treating a patient suffering from an infection caused by Gram positive or Gram negative bacteria, comprising administering an effective amount of a compound of claim 1 to a patient in need thereof.

10. A method of treating a patient suffering from an infection caused by Gram positive or Gram negative bacteria, comprising administering an effective amount of a compound of claim 2 to a patient in need thereof.

11. A method of treating a patient suffering from an infection caused by Gram positive or Gram negative bacteria, comprising administering an effective amount of a compound of claim 3 to a patient in need thereof.

12. A method of treating a patient suffering from an infection caused by Gram positive or Gram negative bacteria, comprising administering an effective amount of a compound of claim 4 to a patient in need thereof.
